(19) United States
(12) Patent Application Publication
(10) Pub. No.: US 2013/0072982 A1
(43) Pub. Date: Mar. 21, 2013

(54) SPINAL ASSEMBLY LOAD GAUGE

(71) Applicant: Peter Melott Simonson, Longboat Key, FL (US)

(72) Inventor: Peter Melott Simonson, Longboat Key, FL (US)

(21) Appl. No.: 13/622,319

(22) Filed: Sep. 18, 2012

Related U.S. Application Data
(60) Provisional application No. 61/536,487, filed on Sep. 19, 2011.

(51) Int. Cl. A61B 17/70; A61B 17/88

(52) U.S. Cl. 606/267; 606/102

(57) ABSTRACT

A load gauge to measure the forces that must be applied to properly position a spinal fixation assembly to one or more vertebrae. In a preferred embodiment, said load gauge is attached to a bone screw. The bone screw is in turn connected to a rod through a connector. After the bone screw is inserted into a vertebrae, the load gauge measures the force that must be applied to properly position the spinal fixation assembly vis-a-vis the vertebrae. The present invention allows surgeons to have more flexibility on how and how much the deformed spine is corrected.

### CROSS-REFERENCE TO RELATED APPLICATIONS

This application claims priority to U.S. Provisional Application No. 61/536,487, filed on Sep. 19, 2011, the disclosure of which is incorporated herein by reference in its entirety for all purposes.

### FIELD OF THE INVENTION

The present invention relates to orthopedics and spinal surgery, more particularly to an instrument that measures the load forces on spinal implants and, especially, bone screws.

### BACKGROUND OF THE INVENTION

Several techniques and systems have been developed for correcting and stabilizing the spine and facilitating a common spinal surgical procedure called fusion. The most widely used systems use a bendable rod that is placed longitudinally along the length of the spine. Such a rod is typically bent to follow the normal curvature of the spine. In such a procedure, a rod is attached to various vertebrae along the length of the spinal column by a number of bone screws inserted into the vertebral body, and, in some cases, by a number of bone screw connectors. When stabilized, the vertebra is decortified where the outer cortical bone is removed to provide a foundation for bone grafts. Over time, these bone grafts fuse the damaged vertebrae together.

A good example of a spinal fixation assembly is the Cotrel-Dubosset/CD Spinal System® sold by Medtronic Sofamor Danek, Inc. Additional details of this technology can be found in U.S. Pat. No. 5,005,562 to Cotrel. As shown in FIG. 1, the introduction of a mono-axial screw 2 in this spinal fixation assembly greatly improves the stability of spinal fixation and allows higher corrective forces to translate and de-rotate the vertebrae 4. A major characteristic of the CD Spinal System® is the tulip bulb connector 6. As shown in FIG. 2, the mono-axial screw 2 only has 2-degrees-of-freedom (arrows) and requires the spinal rod 8 to be pushed, seated, and locked into the connecting slot 10. As such, the mono-axial screw 2 can only move along the rod 8 longitudinally 12 and rotate 14 around its rod 8. With only 2-degrees-of-freedom, the mono-axial screw 2 is limited in its movement for use in a curved spine and in its ability to rotate the vertebrae. When the number of screws is increased, as shown in FIG. 3, a highly overstrained biomechanical system is generated and the ability to control the inter-vertebral forces 16 (dark arrows) at the mono-axial screw 2 and rod 8 interface is severely hampered. In most cases, it is rare that the implants are aligned with the rod and, as such, the spine needs to be forced 17 (light arrow) to the rod 8. During such reduction or translation, all of the inter-vertebral elements are subjected to very high axial loads, shear, stress, and torque forces. It has been reported that the force on the mono-axial screw 2 can be upwards of 300 to 400 Newtons/mm. When such vertebral correction or translation occurs, it will generate such a high force load that bone screw pullout 18 is common. In cases when the bone is strong and healthy, the initial fixation of traditional spinal and orthopedic screws is usually excellent with pullout strength around 150 N/mm. With degenerative cases, pullout strength falls to about 50-60 N/mm.

In referring back to FIG. 1, a good example of a spinal fixation assembly is the TSRH® Spinal System 20 also sold by Medtronic Sofamor Danek Inc. With a unique ability described in U.S. Pat. No. 5,643,263 to Simonson, the TSRH® Spinal System provides vertical adjustability 22. When there is a height difference between the rod 24 and bone screw 26, the TSRH® Spinal System 20 closes this height difference and allows a rod 24 to be situated at variable distances from the spine and/or oriented with a pre-set contour rod regardless of the location of the connector 28. Furthermore, this vertical adjustability 22 reduces the possibility of bone screw pullout 18 on any single bone screw 26 by spreading stress and strain on a bone screw 26 throughout the length of the rod 24 and to other spinal implants. Simple in design, the current TSRH® Spinal System 3Dx 20 allows a surgeon to easily engage a bent spinal rod 24 to the connector 28 with a simple driver 32. Such a driver 32 is turned with enough torque 34 to drive the bone screw 26 deep into the vertebrae 4. In most cases, the TSRH® Spinal System 20 and vertebrae 4 are pulled together without any concern for bone screw pullout 18 strength.

The concept of attaching a pre-contoured rod to a deformed spine was used by Luque and Asher (wires and cables) for scoliosis, as well as, Edwards (threaded connectors) and Steffe (threaded screw posts) for spondylolishthesis. A cabling system described in U.S. Pat. No. 5,395,374 to Miller discloses how cables are used to couple the spine to rods. The tension on the cable is measured with a tensioning tool and the cables are crimped. For a long time, however, there was no bone screw assembly that solved both degenerative and deformity problems. When bone screw assemblies were tried, bone screw pullout was reported to be 5-15%. To compensate, higher torques were applied to bone screws during deformity cases, which sometimes leads to more bone damage. More recently, a multi-degree-of-freedom spinal fixation assembly was developed to allow the connected vertebrae to be translated toward the rod from any distance and at any angle.

A multi-degree-of-freedom spinal fixation assembly was disclosed in U.S. Pat. Nos. 6,309,391 and 7,322,979 to Crandall and commercialized as the TSRH-3Dx® Multi-Planar Adjusting screw. As shown in FIG. 4, this TSRH-3Dx® Multi-Planar Adjusting (MPA™) Screw 36 sold by Medtronic Sofamor Danek Inc. has a long post 38 attached to the pedicle screw 40 with a universal joint 42 connecting the rod 24. As illustrated, this configuration provides 6-degrees-of-freedom (arrows). Briefly, the long post 38 shown in FIG. 5 facilitates simultaneous correction in both the coronal 44 and sagittal 46 planes. When the long post 38 and its pedicle screw 40 are screwed into the pedicle 48, it offers movement 50 through its hinge 52. In combination with the vertical and rotational ability of Simonson's TSRH® 3Dx connector 28, this pivoting post system facilitates simultaneous correction in both the coronal 44 and sagittal 46 planes when pulling the vertebrae 4 to the pre-contoured rod 24. With such a spinal fixation assembly, the force load on a bone screw is reduced by as much as 60%. During vertebral translation, the force load on a MPA™ Screw 36, reduction crimp 54 and driver 56 averages between 20-40 N/mm—well below the bone screw pullout strength in degenerative bone.

This principle of direct vertebral translation to the rod is a powerful tool for deformity correction especially for scoliosis, kyphosis and spondylolisthesis. Although the correction of large and stiff spinal deformities still involves higher implant-vertebra load with higher risk of bone-screw interface damage, implant pullout or vertebral fracture, this risk can be better managed by a multi-degree-of-freedom spinal fixation assembly. With the multi-degree-of-freedom bone screw, the reduction process is still recommended to proceed slowly with only a few turns on a driver, especially in patients with poor-bone quality or osteoporosis. The industry, therefore, needs an accurate and precise measurement of relative force loads applied during vertebral translation that avoids and reduces the risk of possible vertebral fracture and/or bone screw pullout.

In summary, the industry may benefit from an instrument that provides both relative and precise force load measurements during all spinal fixation procedures.

### SUMMARY OF THE INVENTION

The present invention is a spinal fixation assembly that includes a load gauge to measure the force that must be applied to properly position the spinal fixation assembly vis-à-vis spinal vertebrae. In the preferred embodiment, the load gauge can include a mechanical spring or an electrical load cell. Preferably, the load gauge is located within a bone screwdriver and/or reduction crimp where force magnitude changes occur when translating or reducing spinal implants. Since the correction of spinal deformities usually involves higher implant-vertebra load with possible bone-screw interface damage, implant pullout or vertebral fracture, the present invention allows surgeons to have better control over such correction forces and more flexibility on how and how much the deformed spine is corrected.

The present invention makes it possible for spinal translation to be performed in a more incremental and less risky way by applying appropriate forces on each implant and locking them at any point between partial and complete translations. A real time measurement of these implant, inter-vertebral and spinal segment forces allows for a smoother and safer vertebral alignment during reduction or translation.

### BRIEF DESCRIPTION OF THE DRAWINGS

FIG. 1 shows a cross-section view of a vertebral body with spinal fixation assemblies of the type described in U.S. Pat. No. 5,005,562 to Cotrel and U.S. Pat. No. 5,643,263 to Simonson.

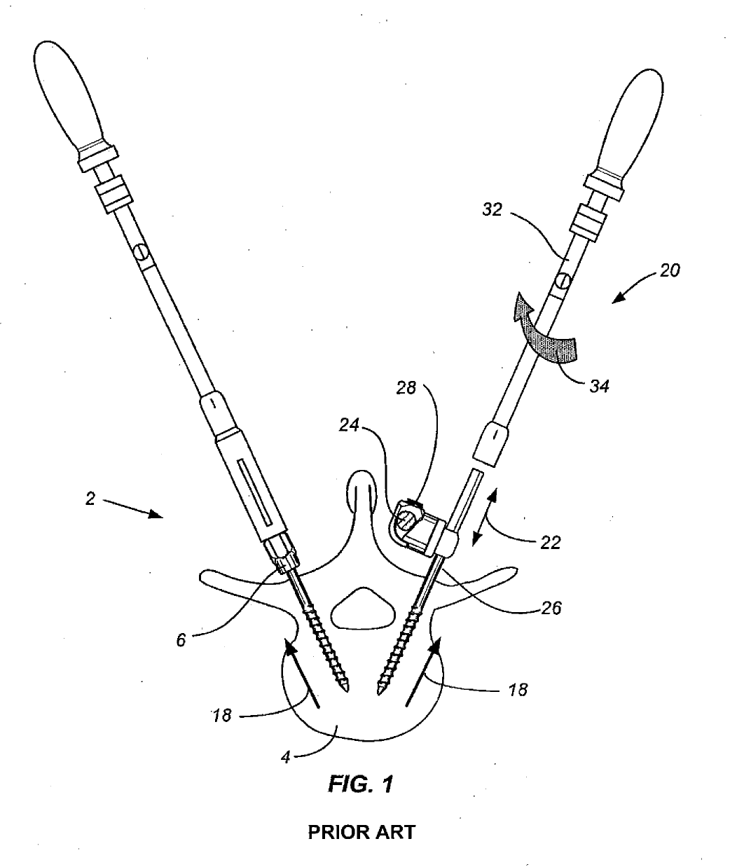

FIG. 2 shows details and degrees-of-freedom of the spinal fixation assembly described in U.S. Pat. No. 5,005,562 to Cotrel.

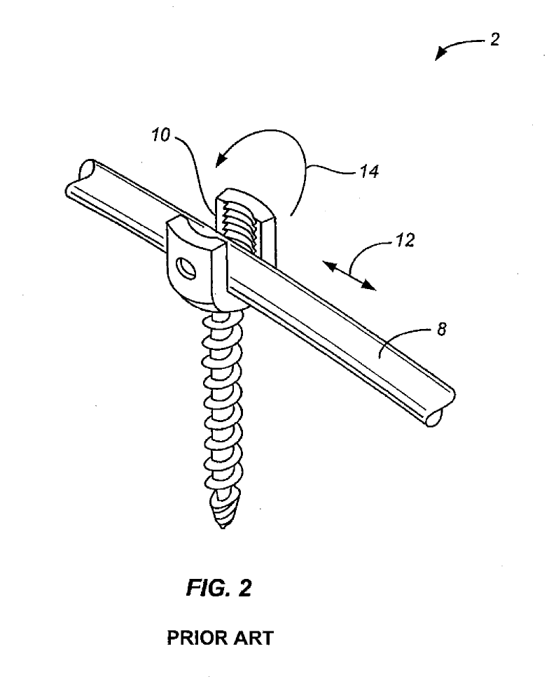

FIG. 3 shows a overly constrained biomechanical spinal system when a spinal rod is pushed, seated, and locked into the spinal fixation assembly described in U.S. Pat. No. 5,005,562 to Cotrel.

FIG. 4 shows details of a multi-degrees-of-freedom bone screw assembly described in U.S. Pat. No. 5,643,263 to Simonson and U.S. Pat. No. 6,309,391 to Crandall.

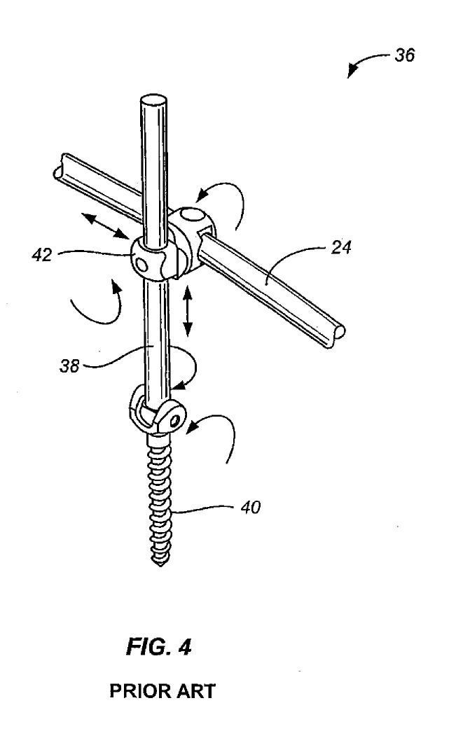

FIG. 5 shows the TSRH-3Dx® Multi-Planar Adjusting (MPA™) Screw multi-degree-of-freedom spinal fixation assembly.

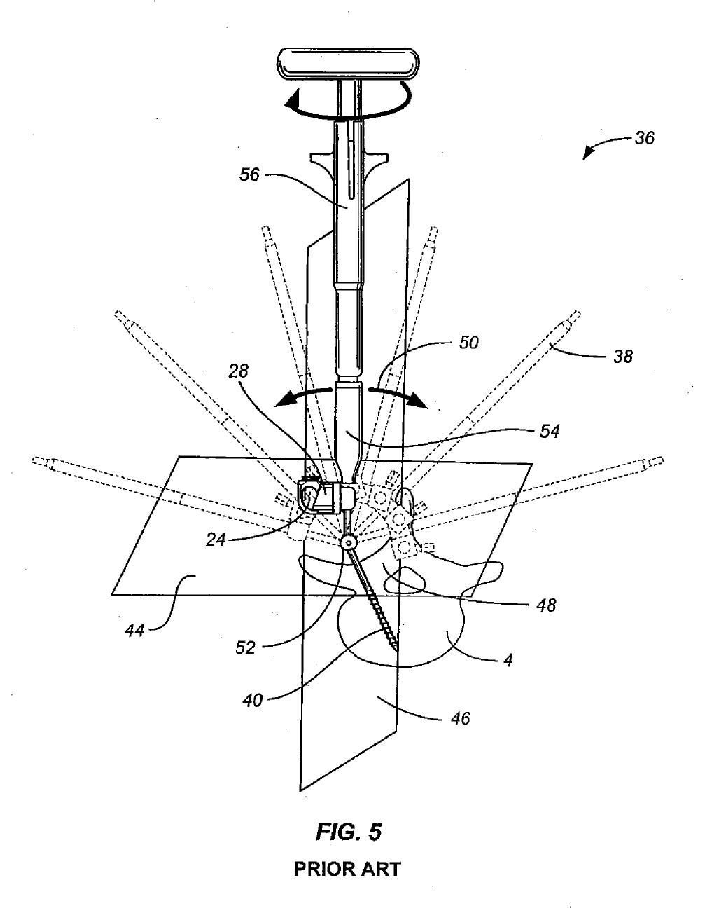

FIG. 6 shows the present invention on the TSRH-3Dx® Multi-Planar Adjusting (MPA™) Screw.

FIG. 7 shows the 6-degrees-of-freedom of the TSRH-3Dx® Multi-Planar Adjusting (MPA™) Screw.

FIG. 8 illustrates the present invention with the TSRH-3Dx® Multi-Planar Adjusting (MPA™) Screw correcting a deformed spine.

FIG. 9 illustrates the inter-vertebral forces during alignment using the TSRH-3Dx® Multi-Planar Adjusting (MPA™) Screw.

FIG. 10 shows the present invention being used with the Cotrel-Dubosset/CD Spinal System®.

FIG. 11 shows a mechanical cross-sectional embodiment of the present invention.

FIG. 12 shows an analog or digital load cell embodiment of the present invention at the driver-reduction crimp interface.

FIG. 13 also shows an analog or digital load cell embodiment of the present invention at the reduction crimp-connector interface.

FIG. 14 illustrates a 6-degree-of-freedom load cell of the present invention.

FIG. 15 shows various embodiments of load cells.

FIG. 16 shows a kyphosis reduction using the present invention with a PC.

### DETAILED DESCRIPTION OF THE INVENTION

### I. GENERAL DESCRIPTION

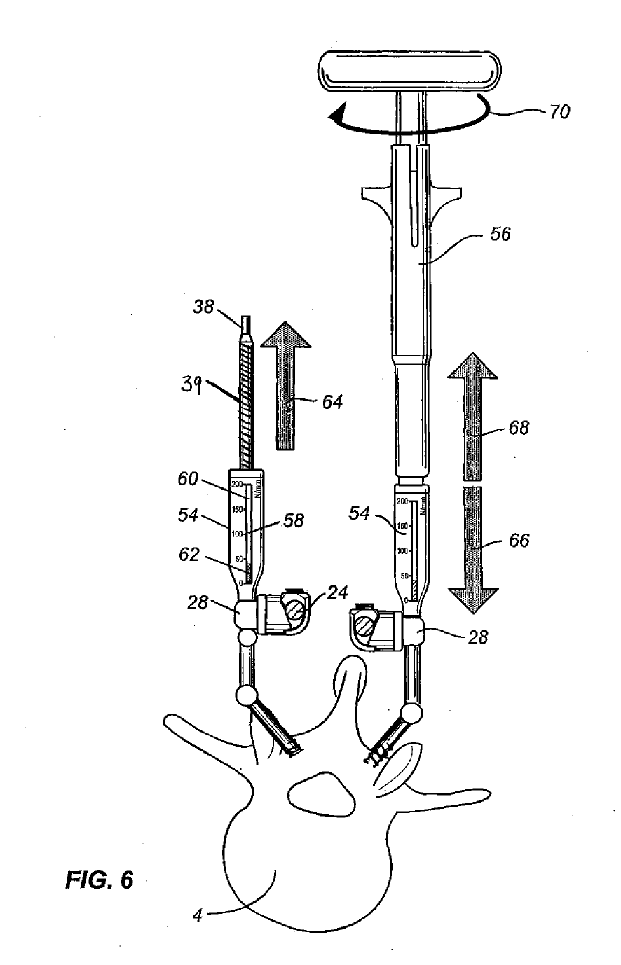

One embodiment of the present invention is illustrated FIG. 6. It is a load gauge 58 placed onto or inside an instrument used with a bone screw assembly. In a preferred embodiment, a reduction crimp 54 with a load gauge 58 threaded at its distal or top end is placed over a threaded 39 long post 38. The lower or smaller end of reduction crimp 54 engages and locks onto a bone screw connector 28. A commercially available reduction crimp called a Provisional Reduction Crimp (Product #8361963) sold by Medtronic Sofamor Danek Inc. is similar to the reduction crimp 54 shown in FIG. 6. In this embodiment, the mechanical, analog or digital components of the load gauge 58 are placed inside the body of the reduction crimp 54 or its driver 56. Furthermore, a visible and graduated Newton/mm scale 60 may be positioned on the surface of the reduction crimp 54. The load gauge 58 may also include a bar-type indicator 62. Such an indicator 62 may preferably have a variety of color bars such as red, yellow and green. The colors may indicate the relative forces experienced by the bone screw assembly and, in particular, the bone screws. For example, red may indicate a high load, yellow may indicate a high but acceptable load and green may indicate a desirable load. In FIG. 6, the long post 38 is threaded upward 64 through the reduction crimp 54. The reduction crimp 54 engages and, as it is screwed downward, pushes down on a bone screw connector 28 drawing the long post 38 upward 64 through the reduction crimp 54. As it does so, it reduces the distance between the vertebrae 4 and a spinal rod 24. By simultaneously turning 70 the driver 56 and reduction crimp 54, the reduction crimp 54 places downward force 66 on the connector 28 and pulls the long post 38 upward 68. The load gauge 58 measures this force as a relative measurement of the load on the spinal implants, especially the bone screws.

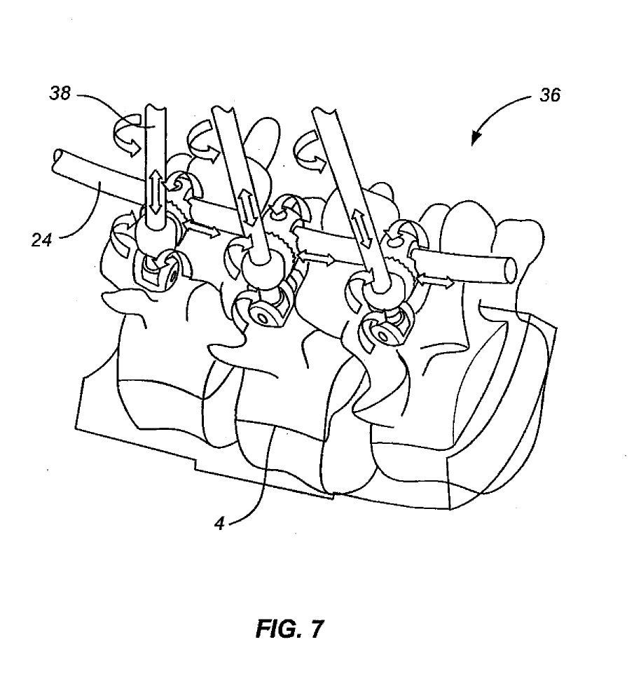

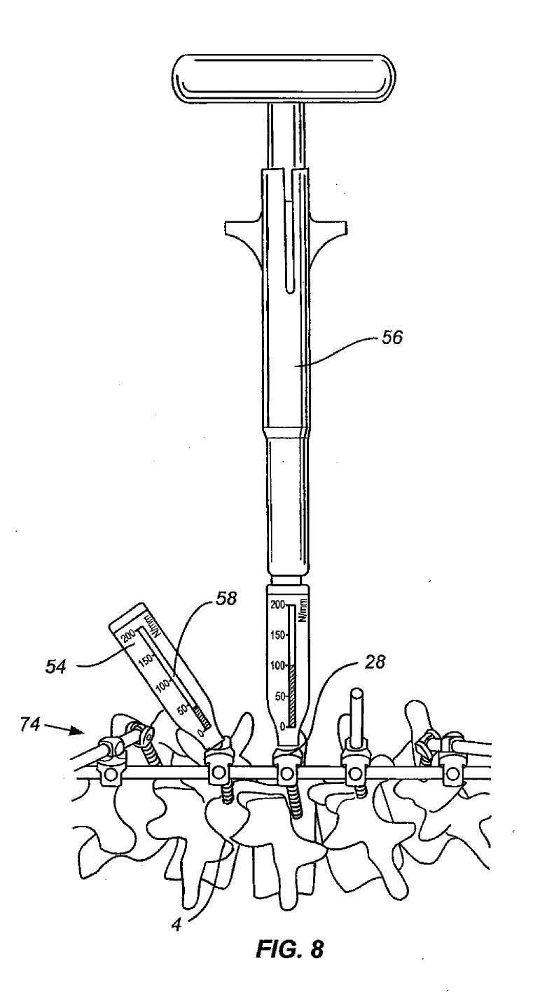

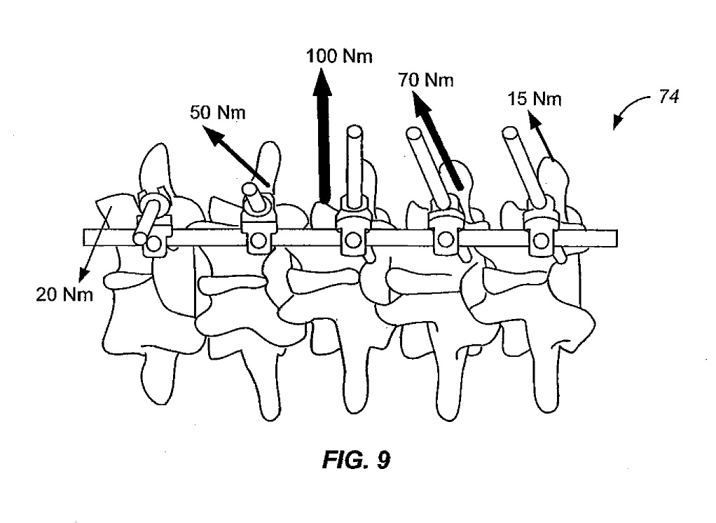

As shown in FIG. 7, a multi-degree-of-freedom bone screw assembly such as the TSRH-3Dx® Multi-Planar Adjusting (MPA™) Screw 36 allows the connected vertebra 4 to be positioned anywhere by pushing, pulling, pivoting and/or turning the long post 38 with respect to the rod 24. The vertebrae positioning offered by the multi-degree-of-freedom bone screw assembly allows the vertebrae to be correctly positioned and unnecessary translation avoided. Even with the multi-degree-of-freedom bone screw, though, the reduction process is still recommended to proceed slowly with only a few turns on a driver, especially in patients with poor-bone quality or osteoporosis. The present load gauge 58 invention illustrated in FIG. 8 can now provide the surgeon with a far more accurate and precise measurement of force loads shown in FIG. 9 and applied during vertebral translation. The present invention may help to avoid and reduce the risk of possible vertebral fracture and bone screw pullout.

In referring back to FIG. 8, the present load gauge 58 invention provides information on the relative forces applied to the bone screw assembly and, in particular, the bone screws. With either a Newton/mm scale or color-coded indicator, the surgeon can now exert the proper torque on the driver 56. Along with multi-degree-of-freedom spinal fixation assembly, the load gauge 58 makes it possible for the translation to be done in an incremental and repeated way with the possibility of applying appropriate force on each implant and locking them at any point between partial and complete translations. In the example shown, a reduction crimp 54 with a load gauge 58 has already partially translated the vertebra allowing the inter-vertebral force to be distributed along the instrumented spinal segment 74. The driver 56 is moved to an adjacent connector 28 where higher inter-vertebral forces shown in FIG. 9 are slowly corrected. Along with the multi-degree-of-freedom spinal fixation assembly, the present load gauge invention may better manage the risk of bone screw pullout because it may allow surgeons to better control the correction forces and provide more flexibility on how and how much the deformed spine can be corrected. As shown in FIG. 9, a surgeon will now know the resultant relative forces on the inter-vertebral elements and, especially, the load, shear, stress and torsion placed throughout the instrumented spinal segment 74.

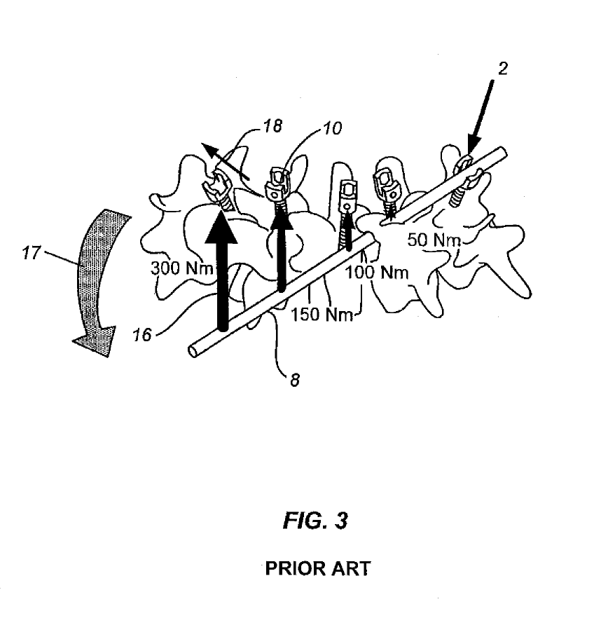

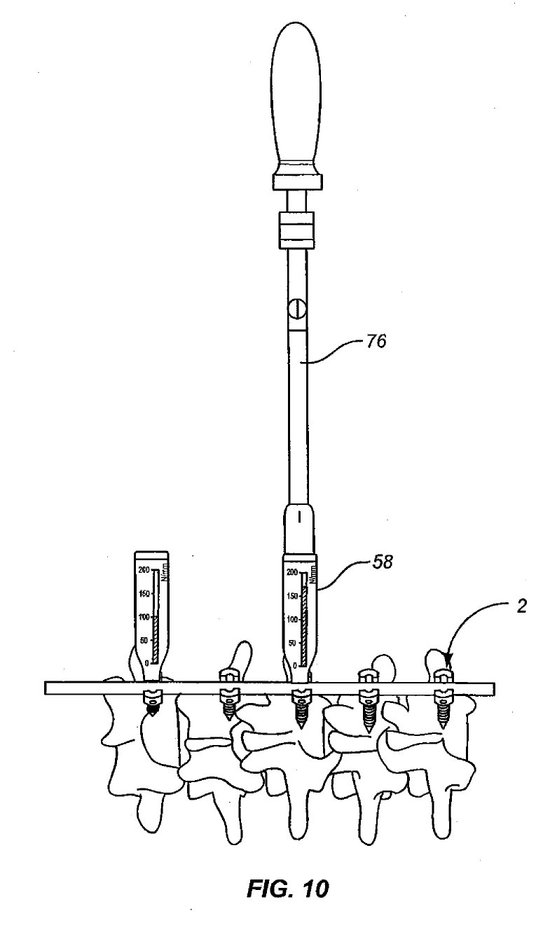

The present invention is particularly important for the mono-axial screws shown in FIG. 3. With mono-axial screws 2, all of the implants have to be fully translated by placing the rod 8 into the screw head slot 10 and screwing down the set-screw, leaving little option for controlling the inter-vertebral forces 16 as well as their distribution along the instrumented spinal segment. Since it rarely is the case that the implants are always aligned with the rod 8, the spine needs to be forced 17 onto the rod 8. Since extra stresses have no benefit to the deformity reduction, all of the inter-vertebral elements are subjected to unnecessary axial loads and shear. Now turning to FIG. 10, the present invention may also be used on a mono-axial bone screw assembly 2. Attached to a mono-axial screwdriver 76, the load gauge 58 may help a surgeon control and limit the forces at the bone-screw interface and the inter-vertebral elements of an overly constrained biomechanical system generated by a mono-axial bone screw assembly 2. Although the force from mono-axial constructs will be a multiple of the force experienced by the multi-degree-of-freedom screws, the present invention may help provide improved correction and clinical benefits in treating patients with stiff deformity as well as treating deformity in patients with compromised bone density using mono-axial bone assemblies.

### II. MECHANICAL LOAD GAUGE

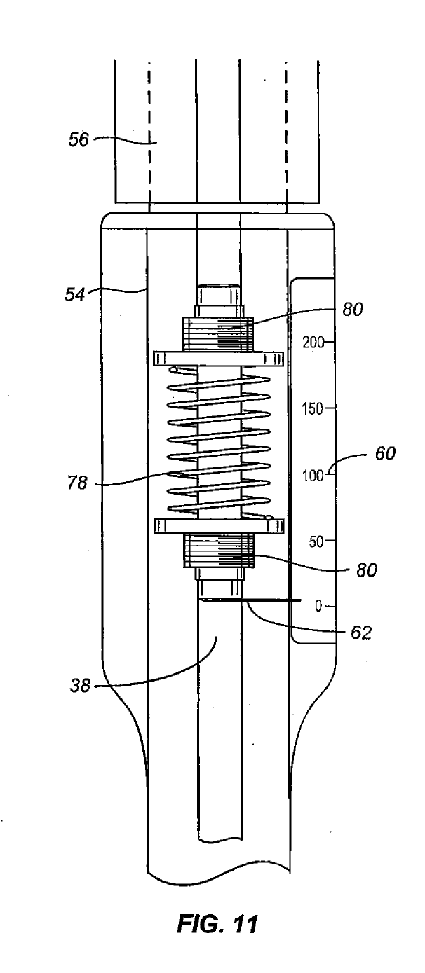

There are numerous preferred embodiments for the load gauge of the present invention. There are generally three types of load gauges—mechanical, analog and digital. As shown in FIG. 11, one preferred embodiment is a gauge having a spring 78 that is compressed or torqued. Such springs 78 are generally made out of hardened or annealed steel or non-ferrous metals such a phosphor bronze, titanium and beryllium copper. A tension/extension, compression or torsion spring 78 may be positioned inside a spinal fixation assembly instrument such as the reduction crimp 54 or driver 56. In its simplest form, the long post 38 of the TSRH-3Dx® Multi-Planar Adjusting (MPA™) screw runs through the spring 78 and is sandwiched between two tension bars 80 that depress a calibrated spring 78. One or both of the tension bars 80 may contain an indicator 62. When depressed, the spring translates its compression or force load to the indicator 62. With a graduated scale 60, the indicator 62 displays a relative force load value. The indicator 62 may also be a color indicator where it exhibits a different color based on the load or stress experienced by the gauge (e.g., red, yellow, green). The spring 78 may also be a calibrated coil that is torqued or turned.

### III. ANALOG/DIGITAL LOAD GAUGE

In a more sophisticated embodiment, an analog or digital instrument may be used as a load gauge. A load cell, for example, is generally a transducer that converts a force into electrical signals. In most load cells, a mechanical component deforms a load gauge that measures the deformation as an electrical signal. The software and electronics of the load gauge converts the voltage of the load cell into a force value that is displayed on the instrument. In a preferred embodiment, the signal is converted by a transducer to a unit of force (e.g., N/mm) and shown on a LED display.

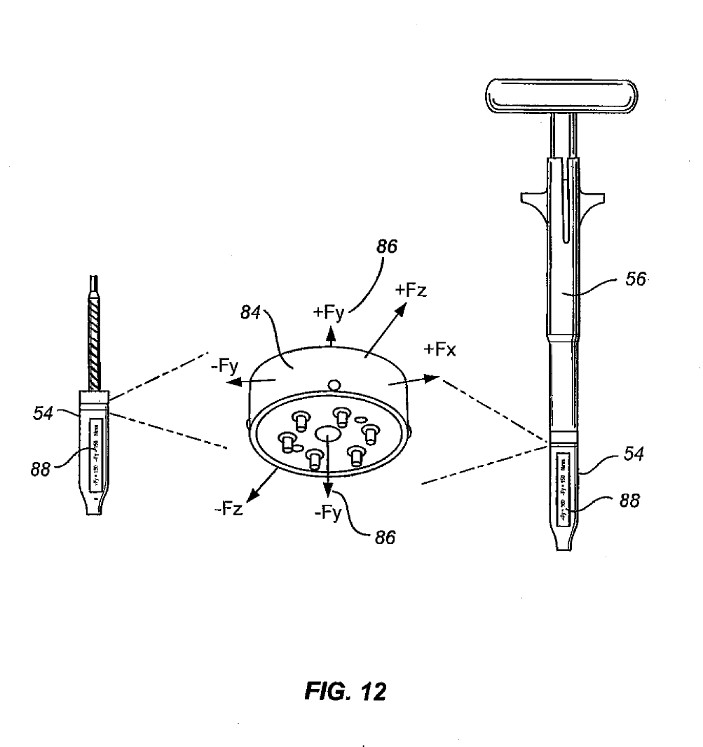

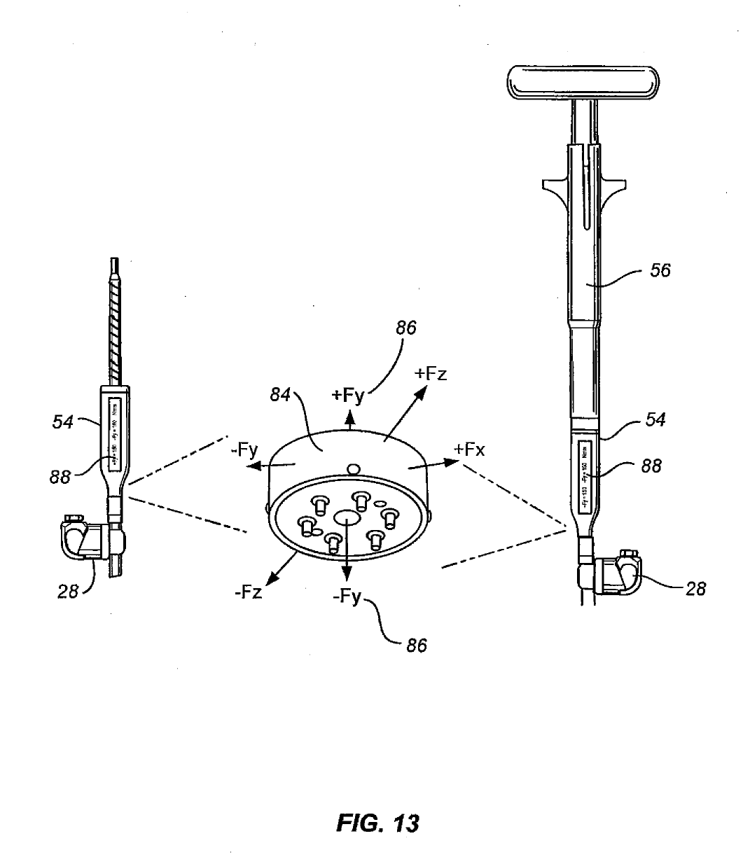

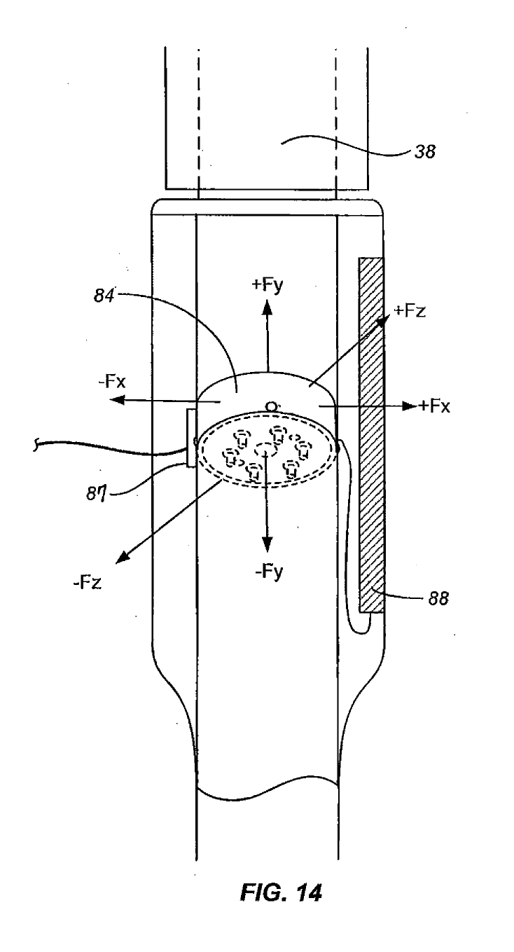

Such load cells are commercially available. One suitable example of a load cell is model 50M31 series from JR3, Inc. of Woodland, Calif. As shown in FIG. 12, these load cells 84 are capable of measuring three orthogonal forces and moments about an x-y-z coordinate system, where the default origin of the coordinate system of each sensor rests in the center of the load cell body. The JR3 load cells are usually monolithic metal devices containing analog and digital systems. Foil strain gauges sense the loads imposed on the sensor. The strain gauge signals are then amplified and combined to become an analog representation of the force loads on the three axis and the moments or torques about the three axes. In most models, the analog data is converted to digital form by electronic systems contained within the sensor. With the reference for all loading data at the geometric center of the sensor, the 50M31 JR3 load cell can be placed at either end of the reduction crimp 54 and the vertical forces 86 can be measured between the reduction crimp 54 and the driver 56 or between the reduction crimp 54 and a connector 28 as illustrated in FIG. 13. The load cell 84 can then measure the static and dynamic compressive or torque forces at the implant interface and convert that data to a digital form. That data can be displayed, for example, by a LED indicator 88 (FIG. 14) on the side of the instrument providing an accurate indication of the load imposed on the load cell 84. The load imposed on such load cell 84 is generally a good representation of the forces being experienced by the spinal implants and, especially, the bone screw.

In a further embodiment, a load cell such as the 100M40M six-axis load cell series, also from JR3, Inc. can be used. Otherwise known as a 6-degree-of-freedom “6-DOF” load sensor, the 6-DOF first measures the in-plane rotation, which corresponds to the rotation within the sagital plane about the flexion-extension “y” axis shown in FIG. 14; the horizontal translation, which correlates to movement along the forward “x” axis; and vertical translation, which correlates to movement along the vertical “z” axis. In this case, the load cell 84 may be a part of the long post 38 either attached directly to it or running through the load cell 84 to measure many of the inter-vertebral forces experienced by the spinal implants. With other features such as internal or external electrical source or battery 87 and, perhaps, a LED indicator 88, the 6-DOF-load cell can provide a surgeon with precise force and moment data instantly.

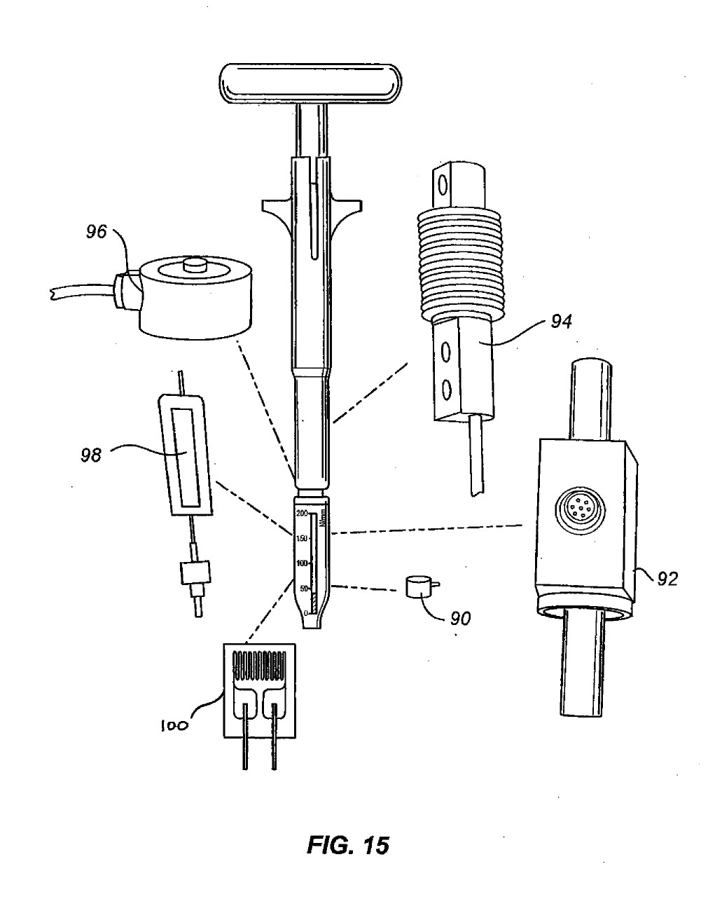

Instead of the JR3 load cells, other sensors shown in FIG. 15 may include piezoelectric force transducers 90, torque transducers 92, bending beam load cells 94, micro strain gauges 96, optical strain gauges 98 and micro strain gauges 100 all sold, for example, by HBM Inc., Marlborough, Mass.

### IV. EXAMPLES

The present invention may be more readily understood by reference to the following examples, which are included merely for purposes of illustration of certain aspects and embodiments of the present invention, and are not intended to limit the invention.

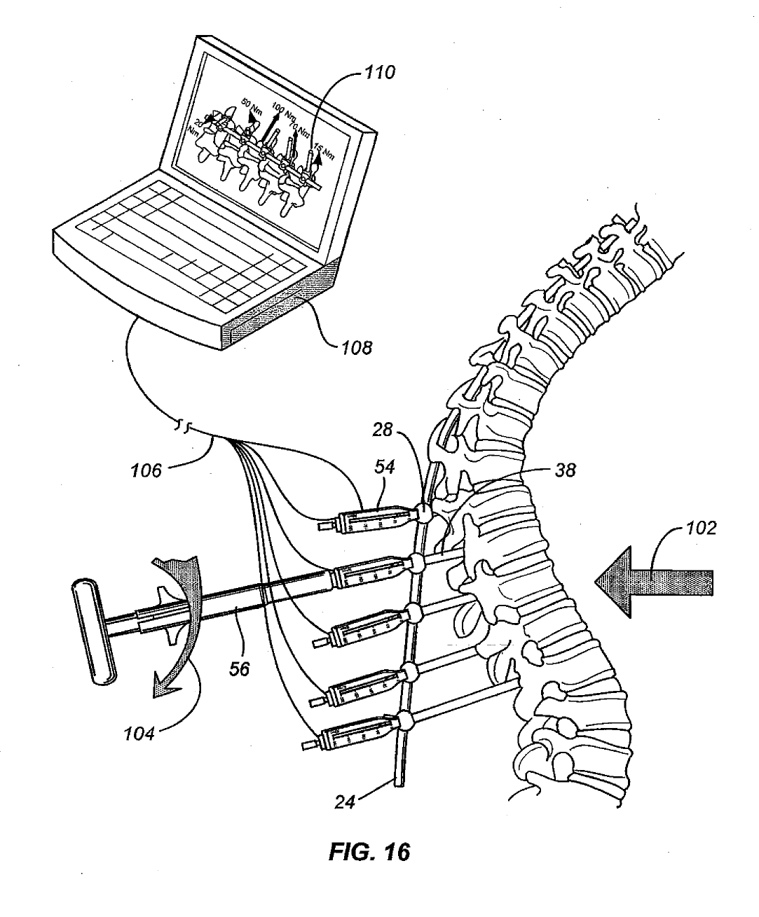

A good example of the present invention is its use in remedying kyphosis (i.e., a curvature of the upper back). FIG. 16 illustrates a spine exhibiting severe kyphoisis deformity. In this case, the surgeon has secured the connectors 28 to the long posts 38 on the upper spine. To correct the kyphosis, the surgeon will pull the lower spine to the pre-contoured rod 24 to straighten the upper spine. As exhibited, the force load 102 to do so may be so high that it can easily pull out any bone screws and damage the vertebrae of the lower spine. Once the lower spine connectors 28 are secured to the long posts 38, the reduction crimps 54 of the present invention are placed onto the long post 38. The driver 56 is turned 104 to advance the reduction crimps 54 and pull the lower spine to the pre-contoured rod 24. Since the forces and moments can now be measured by the present invention, moment and force data from a 6-DOF-load cell can be written into RAM of a computer 108. In particular, the load gauge readings can be transmitted through wire 106 or wirelessly and a force sensor receiver card can read that data and show that data to a surgeon on a screen of a computer 108. A computer receiver card, such as the PCI cards that sold by JR3, Inc., can directly interface with the load cells. A receiver card inside the computer 108 can process the force and moment data and also, provide transformation, vector, thresholds, peak and rate calculations. As shown in FIG. 16, unfiltered data can be collected from the load cell, channeled into a computer and run through data collection program or computer software algorithms such as that sold by Commotion Technology, World Trade Center, San Francisco, Calif. In a very complicated and sophisticated embodiment, an equilibrium position where the external load and the constraint translation or reduction forces of the spine can be balanced. The computer 108 records the position while the load cells continue to measure the residual forces. A 3D digitized image 110 (Microscribe, Solution Technologies, Oelia, Md.) of the spine can be overlaid with the resultant forces. Such a sophisticated embodiment can better manage the risk of bone screw pullout because it now allows surgeons to have better control on the correction forces and provide flexibility on how and how much the deformed spine can be corrected. As shown in FIG. 16, a surgeon can see the resultant forces on 3D digitized images 110 of the spine. As a further alternative, the computer 108 can be connected to a powered or motorized driver 56. Desired load readings or ranges, for example, can be pre-programmed into the computer 108. As measured load readings are received by the computer 108, the pre-programmed load readings can be used by the computer to activate the powered or motorized driver 56 to reach the pre-programmed load readings. The pre-programmed load readings can also be used as a stop to prevent the powered or motorized driver from overtightening.

In the foregoing specification, the invention has been described with reference to specific preferred embodiments and methods. It will, however, be evident to those of skill in the art that various modifications and changes may be made without departing from the broader spirit and scope of the invention. For example, while the load gauge has been described in various mechanical, analog and digital embodiments, those of skill in the art will recognize that alternative devices can be use for similar or different purposes. Accordingly, the specification and drawings are to be regarded in an illustrative, rather than restrictive, sense; the invention being limited only by the appended claims.

What is claimed is:

1. A spinal fixation assembly comprising:
a bone screw for insertion into a vertebrae;
a load gauge connected to said bone screw;
a connector which connects said bone screw to a rod,
wherein said load gauge measures the force that must be applied to said connector and rod to bring them into desired proximity to said vertebrae when said bone screw is inserted into said vertebrae.

2. The spinal fixation assembly of claim 1 wherein said load gauge includes a load cell.

3. The spinal fixation assembly of claim 1 wherein said load gauge includes a spring.

4. The spinal fixation assembly of claim 1 wherein said bone screw includes a screwed end and a long post end wherein said load gauge is attached to said long post end.

5. The spinal fixation assembly of claim 1 wherein said load gauge sends electronic signals to a computer.

6. The spinal fixation assembly of claim 1 wherein said rod connects said bone screw to other bone screws.

7. The spinal fixation assembly of claim 6 wherein one or more of said other bone screws are also connected to load gauges.

8. The spinal fixation assembly of claim 5 wherein said computer controls the distance between said rod and said vertebrae through a powered bone screw driver.

9. A method for measuring forces generated in a spinal fixation assembly comprising the steps of:
attaching a load gauge to a bone screw;
attaching a connector to said bone screw;
attaching a rod to said connector;
moving said connector and rod into desired proximity to said vertebrae; and
using said load gauge to measure the force that needs to be applied to said connector and rod to bring them into desired proximity to said vertebrae.
